(19) United States
(12) Patent Application Publication
(10) Pub. No.: US 2011/0098232 A1
(43) Pub. Date: Apr. 28, 2011

(54) Methods For The Selective Treatment Of Tumors By Calcium-Mediated Induction Of Apoptosis

(76) Inventor: Charles E. Zeilig, Thornton, CO (US)

(21) Appl. No.: 12/911,723

(22) Filed: Oct. 25, 2010

Related U.S. Application Data
(63) Continuation-in-part of application No. 10/558,079, filed on Nov. 22, 2005.
(60) Provisional application No. 60/475,063, filed on May 30, 2003.

(51) Int. Cl. A61K 38/13; A61P 35/00; A61K 38/02

(52) U.S. Cl. 514/19.2

(57) ABSTRACT

Available evidence indicates that tumor cells exhibit consistent abnormalities in Calcium influx and intracellular storage of sequestered Calcium when compared to normal cells. The present invention provides clinical methods by which such differences are exploited to induce Apoptosis selectively in tumor cells while sparing normal cells. These methods are based upon employing drugs that, acting alone or in synergistic combinations, produce an increase in intracellular Calcium loading such that either or both of two major Apoptotic pathways are triggered to produce selective killing of malignant cells. Since the invention is based upon fundamental cell cycle requirements, to the extent that Calcium handling abnormalities are a general characteristic of the malignant state, the methods presented here are widely applicable regardless of tissue of origin and degree of cellular de-differentiation.

This application is a continuation in part of United States patent application Ser. No. 10/588,079, filed Nov. 22, 2005, entitled “Methods For the Selective Treatment of Tumors by Calcium-Mediated Induction of Apoptosis,” which claims priority to U.S. provisional application Ser. No. 60/475,063 entitled “Methods For the Selective Treatment of Tumors by Calcium-Mediated Induction of Apoptosis,” filed May 30, 2003; the entire disclosures of which are hereby incorporated by reference. Any disclaimer that may have occurred during the prosecution of the above-referenced applications is hereby expressly rescinded, and reconsideration of all relevant art is respectfully requested.

### FIELD OF THE INVENTION

This present invention is in the field of medical therapeutics, more particularly in the field of clinical treatment of malignancy. The methods allow a broad range of human tumors to be treated by selectively inducing apoptosis. Apoptosis is induced in tumors by disrupting intracellular calcium distribution in a manner that leaves normal growing or non-growing cells unharmed.

### BACKGROUND OF THE INVENTION

### A. Observations Concerning Cell Cycle Control Mechanisms

Calcium (abbr. Ca, Ca⁺⁺as freely diffusable) is an essential requirement for the growth of normal and malignant mammalian cells (Whitaker, M. and Patel, R. (1990) Development 108:525-542; Lu, K. P. & Means, A. R. (1993) Endocrine Rev. 14, 40-58; Means, A. R. (1994). FEBS Let. 347:1-4; Hepler, P. K. (1994) Cell Calcium 16:322-330; Whitaker, M. (1997) Progress in Cell Cycle Research 3, 262-269; Whitaker, M. and Larman, M. G. (2001) Cell & Developmental Biology 12:53-58). Entrance into the cell cycle program (i.e., the transition from G0 to G1) is initiated by various growth factors that trigger a massive and sustained influx of Ca++ (Berridge, M. J. (1995) Bioessays 17:491-500). The cellular response to this unusually prolonged influx of Ca++ is characterized by precise adjustment of isoform-specific expression of, for example, Calmodulin (abbr. CAM; Brooks-Frederich, K. M. et al. (1993) Exp. Cell Res., 205:412-415; Pinol, M. R., et al. (1988) FEBS Letters 231:445-450), Sarco-Endoplasmic-Reticulum Ca-ATPase (abbr. SERCA; Petzelt, C. and Auel, D. (1977) Proc. Natl. Acad. Sci. U.S.A. 74:1610-1613; Waldron, R. T et al. (1994) J. Biol. Chem. 269:11927-11933; Simon, V. R. and Moran, M. F. (2001). Cell Proliferation 34:15-30), and Plasma Membrane Ca-ATPase (abbr. PMCA; Afroze, T. and Husain, M. (2000) J. Biol. Chem.; 275: 9062-9069). These changes occur prior to the point at which cells become committed to the cell cycle program (the so-called Restriction Point, abbr. RP; Pardee, A. B., (1974). Proc. Natl. Acad. Sci. U.S.A. 71:1286-1290) and coincides with the time at which extracellular (abbr. EC) growth factors are no longer required.

Most oncogenic mutations function to produce a persistent stimulus at various points in the pathway that converges on the final event that allows cells to pass through the RP. Such a persistent stimulus would be expected to lead to abnormal expression of many proteins that control the distribution and function of Ca++. Indeed, many such abnormalities in transformed cells have been reported as exemplified by alterations in Plasma Membrane Ca gates (Chen, C. et al. (1988) Science 239:1024-1026), binding proteins (MacManus, J. P. et al. (1989) Adv. Exp. Med. & Biol. 269:107-110; Kligman, D. and Hilt, D. C. (1988) Trends Biochem, Sci. 13:437-443), CAM (Blum, J. K. et al. (1989) Adv. Exp. Med. & Biol. 269:121-125; Van Eldik, L. J. et al. (1989) Adv. Exp. Med. & Biol. 269:111-120), CAM-dependent protein kinase IV (Takai et al. (2002) Cancer Let. 183:185-193), and phosphatidylinositol 3-kinase (Vivanco, I. And Sawyers, C. L. (2002) Nat. Rev. Cancer 2:489-501). Although detailed surveys of the quantity and types of SERCA isoforms expressed in malignant cells have yet to be undertaken, it is commonly observed that many transformed cells are capable of proliferation in reduced Ca-containing growth media yet still exhibit an absolute Ca requirement for proliferation. These observations suggest that both Ca influx and sequestration capacity may be upregulated as a consequence of the malignant state. Reports of markedly increased quantities of Ca-buffering proteins in tumor cells (e.g. the S-100 and oncomodulin protein families) strongly suggest that normal Ca sequestration reservoirs operate in tumor cells at or nearly at their full storage capacity in contrast to non-malignant growing cells.

Restriction of EC Ca++ availability during middle G-1, middle S-phase, and Prophase does not impede traverse of cells through these phases. Rather, cells become arrested in the immediately following phases of late G1, G2, and mid-Metaphase (Whitfield, J. F. et al. (1976) In Vitro 12: 1-18; Tupper, J. T., et al. (1980) J. Cell Physiol. 104:97-103; Zeilig, C. E. (1978) In Grant Application to NIH, unpublished). An experimental example of the effect of restricting EC Ca++ availability on the transition of cells from late S-Phase to G-2 in synchronized Novikoff Hepatoma cells is shown in FIG. 1. This experiment shows that when EC Ca++ was restricted, cells closer to prophase than the length of G2 entered prophase normally. On the other hand, cells residing at any point within S-phase failed to enter prophase. It is now well established that passage of cells through late G1, G2, and mid-Metaphase is dependent on Ca++ and functional CAM (Rasmussen, C. D. and Means, A. R. (1989) EMBO J. 8:73-82; Welsh, M. J. et al. (1978) Proc. Natl. Acad. Sci. U.S.A. 75:1867-1871; Lu, K. P. & Means, A. R. (1993) Endocrine Rev. 14, 40-58; Zeilig, C. E. et al. (1974) In 2^{nd} Int. Conf. Cyclic AMP, Vancouver, Canada (Tha-10a); Zeilig, C. E. et al. (1976) J. Cell Biol. 71:515-534; Friedman, D. L. et al. (1976) In Cyclic Nucleotides and the Regulation of Cell Growth. Dowden, Hutchinson, and Ross, Inc., pp. 57-79). These are the same phases that do not require the presence of EC Ca++.

### B. Integration of Cell Cycle Regulatory Systems

These observations have led to an integrated model of cell cycle control in which specific protein isoforms for controlling Ca distribution becomes established as a prerequisite for passage through RP and which regulates passage of cells through the remaining cell cycle phases. This model provides a detailed explanation of the mechanism by which passage through the post RP phases of the cell cycle is linked to sequential storage of Ca in one phase, followed by release of Ca++ in the immediately next phase. This proposed system is essential for cell cycle traverse in both normal and malignant cells. Moreover, since cell cycle progress is not growth factor-dependent from late G1 forward, this pattern of storage and release must therefore depend upon intracellular control mechanisms. While not essential to the validity of the present invention, and while not limiting the scope of the invention in any manner, this model has proven extremely useful in providing a theoretical framework within which a wealth of published observations can be interpreted and explained.

The timing of changes in Ca storage and release appear to correlate precisely with the expression of specific active forms of cyclin kinases, the engine that drives cell cycle traverse (reviewed in Nurse, P. (2000) Cell 100:71-78). Reciprocal changes in cyclic nucleotide levels are closely correlated with the sequential expression of specific active cyclin kinase complexes. Moreover, cGMP and cAMP levels (Zeilig, C. E. et al. (1972) J. Cell. Biol. 55:296a ; Zeilig, C. E. et al. (1976) J. Cell Biol. 71:515-534; Zeilig, C. E. and Goldberg, N. D. (1977) Proc. Natl. Acad. Sci. U.S.A. 74:1052-1056) correlate with Ca storage and release phases, respectively. Precise measurements of cyclic nucleotide levels, cyclin B kinase activity, and cell cycle kinetics have shown that reciprocal fluctuations in cAMP and cGMP exhibit a near square wave pattern of change, occur at the beginning of a particular phase within minutes of cyclin kinase activation, and are maintained in a stable state for the complete duration of that phase (Zeilig, C. E. and Goldberg, N. D. (1977) Proc. Natl. Acad. Sci. U.S.A. 74:1052-1056; Zeilig, C. E. and Langan, T. A. (1980) Biochem. Biophys. Res. Comm. 95:1372-1379). Once cells pass the RP, both cyclic nucleotide fluctuations and Ca requirements for cell cycle traverse appear to be highly conserved throughout the eukaryotic kingdom regardless of their original differentiated origins.

These changes in cyclic nucleotides and Ca requirements reflect the existence of recurring major regulatory/metabolic shifts during the cell cycle. Among such regulatory shifts, there must occur coordinate changes in the activities of plasma membrane and endoplasmic reticulum Ca gates and pumps in order to explain the periodic uptake and release of Ca. Direct evidence indicates that CAM-dependent processes dominate and are required during Ca-release phases (reviewed in Rasmussen, C.D. and Means, AR (1989) EMBO J. 8:73-82; Lu, K. P. & Means, A. R. (1993) Endocrine Rev. 14, 40-58). Several lines of evidence suggest that specific isoforms of Protein Kinase C (abbr. PKC) may be the dominant Ca effector(s) during Ca storage phases (Fishman, D. D. et al. (1998) Int. J. Oncol. 12:181-186; Black, J. D. (2000) Front. Biosci. 5:D406-D423; Tang S. et al. (1997) J. Biol. Chem. 272: 28704-28711; Villalonga, P. et al. (2002) J. Biol. Chem. 277:37929-37935.)

C. Relationship between Apoptosis and Cellular Calcium Levels

Initiation of Apoptosis (programmed cell death) is generally thought to be triggered by two different pathways: a) extracellular through activation of the Tumor Necrosis Factor (abbr. TNF) or FAS Ligand (abbr. FASL) family of receptors (Cleveland, J. L. and Ihle, J. N. (1995) Cell 81:479-482; Nagata, S. and Goldstein, P. (1995) Science 267:1499-1456), and b) intracellular through stress or chromosomal damage, p53-mediated pathways (White, E. (1966) Genes Dev. 10:1-15). Both pathways appear to converge or intersect downstream at the level of both the caspase 3 death-effector protease, at the mitochondrial Permeability Transition Pore (abbr. PTP), and possibly the SER (Marzo, I., Brenner, C., Zamzami, N., Susin, S. A., Beutner, G., Brdiczka, D., Remy, R., Xie, Z._H., Reed, J. C., and Kroemer, G. (1998) J. Exp. Med. 187:1261-1271; Halestrap, A. (2000) The Biochemist 22:19-24; Cory, S. and Adams, J. M. (2002) Nat. Rev. Cancer 2:647-656).

Excessive Ca has been shown to induce apoptosis in several different cell types (Nicotera, P. and Orrenius, S. (1998) Cell Calcium 23:173-180). Under other conditions, exposure of cells to various agents thought to increase cytosolic Ca++ may antagonize apoptotic responses. These opposing reports have generated considerable controversy and confusion in the field. This area has been reviewed recently by Berridge (Berridge, M. J. et al. (2000) Nat. Rev. Mol. Cell. Biol. 1:11-21) and it has been speculated that whether Ca++ is pro- or anti-apoptotic in a given cell may depend on the interplay between SER and mitochondrial pools of Ca. According to the model presented here, the role of Ca in apoptosis would vary according to the complex ratio of all plasma membrane, SER, and mitochondrial Ca pumps and gates in any one cell type. Moreover, it is this ratio that must be adjusted for cells to pass RP. Thus, in a single population of proliferating cells, some Ca-enhancing stimuli would be predicted to promote apoptosis in only one fraction of cells, depending on whether they resided in pre- or post-RP cell cycle phases. One of the consequences of malignancy would be to increase the differences in this ratio compared to non-cycling cells. Given the reported abnormalities in the amounts of Ca-handling proteins between cycling normal and transformed cells, these differences can be exploited clinically.

Of particular relevance to this application is the recent discovery of so-called “Death-Associated” Protein Kinase (abbr. DAPK; Cohen, O. et al. (1997) EMBO J. 16:998-1008; Kawai, T. et al. (1999) Oncogene, 18:23:3471-3480). This enzyme family is activated by association with Ca++/CAM and appears to be involved as a critical intermediary in the TNF/FASL and JNK apoptotic pathways (Cohen, O. et al. (1999) J. Cell Biol. 146:141-148). This raises the question as to why this enzyme is not activated during CAM-dependent cell cycle phases. Somehow DAPK must normally be prevented from exposure to cytosolic Ca++ concentrations required to stimulate CAM-requiring cell cycle traverse processes. It is known that DAPK has a subcellular distribution restricted by association with cytoskeletal actin microfilaments (Tereshko, V. et al. (2001) Nature Structural Biol. 8:10:899-907) Likewise, cellular SER's appear to have fixed and specific locations within the cell (see Berridge, M. J. et al. (2000) Nat. Rev. Mol. Cell. Biol. 1:11-21). It is hypothesized here that DAPK is prevented from being activated during phases known to be dependent on Ca++/Calmodulin function owing to a fixed association with a third class of SER (called “Guard” or GSER in FIG. 2), located deep within the cell and maximally separated from plasma membrane Ca++ gates. In normal cells, the balance of HCSER to various Ca++ influx channels and to PCMA is such that GSER remains fairly devoid of sequestered Ca++. In tumor cells that have lost the ability to down-regulate either the initial Ca efflux seen upon growth factor exposure or that have lost the ability to down-regulate downstream pathways between Ca influx and induction of new SERCA, at regulatory equilibrium, GSER would be expected to exhibit both higher total storage capacity and a higher degree of Ca++ filling, compared to normal cells. This equilibrium point is set by the requirement of the storage/release regulatory system to control cytosolic Ca++ levels over the range of no CAM stimulation to PKC stimulation thresholds. Accordingly, tumor cells must establish this new equilibrium in order to proliferate continuously. Only when the total Ca++ storage capacity of GSER was exceeded would enough Ca++ leak out (Logan-Smith, M. J. et al. (2001) J. Biol. Chem. 276: 46905-46911) in the immediate vicinity of DAPK to trigger an apoptotic response.

Under certain experimental conditions, preventing stimulation of Ca release from SER by physiological second messengers (e.g. Sphingosine-1-Phosphate, abbr. S-1-P; and cyclic Adenosine Diphosphate Ribose, abbr. CADPR, Han, M. -K., Cho, Y. -S., Kim, Y. S., Yim, C. -Y., and Kim, U. -H. (2000) J. Biol. Chem. 275:20799-20805) has been reported to induce apoptosis suggesting the possibility that over-filling of Ca SER sites may be pro-apoptotic. Cyclic GMP may also contribute to stimulation of this pathway. Cyclic GMP acting through PKG appears to promote Ca++ sequestration by interfering with the ability to release Ca++ from the SER (Komalavilas, P. and Lincoln, T. M. (1994) J. Biol. Chem. 269:8701-8707) and in at least some cell types by stimulating SERCA (Cornwall, T. L. et al. (1991) Mol. Pharmacol. 40:923-931). In addition to this mechanism, cGMP may also coordinately activate a second step in the same pro-apoptotic pathway by Protein Kinase G (abbr. PKG)-mediated activation of Mitogen Activated Extracellular Receptor Kinase Kinase 1 (abbr. MEKK1) followed by downstream activation of the JNK pathway (Soh et al., (2001). J. Biol. Chem. 276:16406-16410). Under these conditions, increased accumulation of cGMP would be pursuant to excessive filling of SER, subsequent leakage of Ca++ and activation of CAM-sensitive Nitric Oxide Synthase (abbr. NOS; Lee, S. -J. and Stull, J. (1998) J. Biol. Chem. 372:27430-27437), resulting finally in the generation of Nitric Oxide (abbr. NO), and stimulation of soluble Guanylyl Cyclase activity (abbr. SGC; Arnold, W. P., Mittal, C. K., Katsuki, S. and Murad, F. (1977) Proc. Natl. Acad. Sci. U.S.A 74:3203-3207). Likewise, depending on the extent of expression of mechanisms to pump Ca++ out of the cell (e.g. PMCA and/or Na/Ca Antiporter, (abbr. NCX), inhibiting SERCA alone might produce sufficient increases in cytosolic Ca++ to trigger apoptosis in some cell types. While not yet proven, this interpretation is strikingly supported by the recent results of Srivastava et al. reporting on the apoptotic effects of the SERCA inhibitor thapsigargin in Jurkat T lymphocytes (Srivastava, R. K. et al. (1999) Mol. Cell. Biol. 19:5659-5674).

Apoptosis can thus be triggered by over-filling of normal sequestration sites to the point at which leakage of Ca++ out of the “Guard” SER would be sufficient to activate DAPK and MEKK/JNK intermediates of the TNF/FASL pathway. It is interesting to note that two drugs under development as tumor-selective inducers of apoptosis act by enhancing the accumulation of cGMP (Sulindac™ sulfone and derivatives; Soh et al., (2001) J. Biol. Chem. 276:16406-16410) and preventing the synthesis of S-1-P (Phenoxodiol™). This apparent selectivity for tumor cells over normal cells can be explained solely in terms of the effect of these drugs on Ca distribution and the presumed excessive Ca++ sequestering capacity contained within malignant cells. Such an explanation has not been proffered by the developers of either of these drugs. Further support for this interpretation has recently been provided by the work of Pitari and coworkers (Pitari, G. M. et al. (2001) Proc. Natl. Acad. Sci. U.S.A. 98:7846-7851; Pitari, G. M. et al. (2003) Proc. Natl. Acad. Sci. U.S.A. 100: 2695-2699). These investigators have shown that prolonged elevation of cGMP levels, through receptor-mediated stimulation of plasma membrane GC, results in direct, cGMP-dependent, activation of a PM Ca++ gate and EC Ca++-dependent inhibition of cell cycle traverse in cultured human carcinoma cells, results predicted by the cell cycle regulatory model referred to above and consistent with the hypothesis that malignant cells are unusually sensitive to excess Ca++ loading.

Prolonged cellular accumulation of Ca++ may also play a role in the p53 apoptotic pathway by stimulating the mitochondrial release of Cytochrome c. As SER pools become filled to capacity, mitochondria can act to buffer increases in free cytosolic Ca++. Available evidence suggests that mitochondrial Ca++ overload promotes formation of the PTP, thus leading to release of cytochrome C and stimulation of death effector caspases (Halestrap, A. (2000) The Biochemist 22:19-24; Kantrow, S. P. and Piantadosi, C. A. (1997) Biochem. Biophys. Res. Comm. 232:669-671).

The unexpected result taught in this disclosure is that it is possible to activate both intracellular and extracellular apoptotic pathways in a common way by pharmaceutically inducing a state of excess Ca++ loading within cells. Thus, the odds of overcoming mutational defects in either pathway and triggering an apoptotic response in a population of malignant cells are effectively doubled. Differences in the extent of SER filling between normal and malignant cells can then provide a window of therapeutic opportunity. Clinical strategies based on either stimulation of excess Ca sequestration or selective inhibition of SERCA will be effective in promoting tumor-specific apoptosis.

Insofar as tumor cells exhibit abnormal levels of various Ca handling proteins, they are quantitatively but not qualitatively different from normally proliferating cells. Thus, while there appears to be a general increase in the expression of specific isoforms of Ca handling proteins, this increase must still produce a precisely balanced equilibrium that allows even malignant cells to execute the cytosolic fluctuations in free Ca concentrations required to effect the sequential activation of CAM- and PKC-dependent processes, sequential storage and release of Ca, and ultimately passage through the growth factor-independent phases of the cell cycle. Because Ca++ is used to control many different critical physiological responses, pharmacological manipulation of Ca fluxes in humans would be fraught with undesirable side effects. By subverting various physiological mechanisms for controlling Ca distribution, it is therefore possible to trigger a differential apoptotic response in tumor cells while sparing non-malignant cycling cells. The invention provides new anti-tumor drug targets and further provides novel strategies for minimizing drug side effects by synergistically combining unexpected classes of drugs to achieve the desired therapeutic outcome.

Any references cited in this application are expressly incorporated herein by reference in their entirety.

### SUMMARY OF THE INVENTION

The instant application provides novel methods for triggering apoptosis selectively by exploiting the differences in Ca handling between malignant cells and normal cells. This is achieved by subverting the normal Ca++ distribution control mechanisms and feedback loops. In order to minimize unwanted side effects, the invention provides therapeutic methods that involve the use of two or more drug combinations that interact synergistically, thus allowing lower drug levels to be used clinically. In one aspect, the disclosure teaches a method for treating a patient with a tumor, the method involves promoting over-filling or prolonging the Ca sequestration state. In one embodiment, the disclosure teaches a method for treating a patient with a tumor, wherein the method involves promoting prolonged elevation of cytosolic Ca++ by preventing normal efflux or sequestration mechanisms in order to trigger “hidden” or occluded apoptotic pathways. By over-filling various cellular Ca++ storage compartments, apoptosis is triggered. Ultimately, once the capacity of these depots is exceeded, DAPK and mitochondrial apoptotic pathways are initiated. Each of these cases requires an unusually large and sustained increase of Ca++ in either general or specific cytosolic compartments as the proximal stimulus to apoptosis.

The application discloses several embodiments of therapies that involve the subversion of the normal Ca++ distribution control mechanisms and feedback loops. Each involves circumventing normal cellular feedback mechanisms for lowering total cellular Ca++. Ca++ efflux is affected by two types of pumps within the plasma membrane: a) a low capacity, high affinity Ca++ ATPase; the so-called family of PMCA's; and, b) a high capacity, low affinity NCX (the Na/Ca Antiporter).

In one embodiment, excess filling is promoted of all Ca++-sequestering depots within the cell, thereby exceeding the capacity of all such depots. This mimics the cell's own way of triggering apoptosis. In this embodiment, a patient with a tumor is treated with inhibitors of SER gates or stimulators of SER pumps in combination with inhibitors of PMCA and NCX. Because malignant cells normally function closer to full Ca++ storage capacity compared to normal cells, lower concentrations of these types of drugs act synergistically to promote Ca++ leakage and apoptosis in a shorter time in tumor cells compared to normal cells.

In one embodiment, the release of all sequestered Ca++ is promoted while simultaneously inhibiting efflux in order to deliver elevated Ca++ to cytoplasmic apoptotic locales (e.g. DAPK). In this embodiment, a patient with a tumor is treated with Ca++ release agonists, including but not limited to NAADP, cADPR, and rynaodone analogs, in combination with PMCA and NCX antagonists. A concentration advantage over normal cells occurs because in malignant cells if the SER in general, and GSER in particular, operate near capacity, very low concentrations of Ca-releasing drugs produce a larger total quantity of Ca to NOS and DAPK sites.

In one embodiment, excess cellular loading of Ca++ is promoted in a patient with a tumor by activating any of several PM Ca gates while simultaneously and synergistically blocking all efflux pumps.

In one embodiment, a state of mitochondrial Ca overload is promoted in a patient with a tumor in order to trigger PTP formation, release of cytochrome C, and ultimately apoptosis.

In one embodiment, restoration of abnormal expression of Ca++ or apoptotic regulatory systems is promoted in a patient with a tumor in conjunction with any therapy provided in the preceding embodiments.

### BRIEF DESCRIPTION OF THE DRAWINGS

FIG. 1 lists known protein isoforms that control Ca++ distribution within cells, along with exemplary drugs of regulatory molecules known to influence the activity of these proteins. In FIG. 1, PM is the abbreviation for plasma membrane; NCX is the abbreviation for Na/Ca Antiporter; SER is the abbreviation for Smooth Endoplasmic Reticulum; SERCA is the abbreviation for Sarco-Endoplasmic-Reticulum Ca-ATPase; DAG is the abbreviation for diacylglycerol; SOC is an abbreviation for store-operated Ca gate; ROC is an abbreviation for receptor-operated Ca gate; VOC is an abbreviation for voltage-operated Ca gate; HTRP is an abbreviation for human homolog of Drosophila Transient Receptor Potential Channel; pp60^{c-src} is an abbreviation for the cellular analog of Sarcoma Virus Tyrosine Knase; PTP is an abbreviation for permeability transition pore; ΔΨₘ is an abbreviation for mitochondrial membrane potential; and Ryr is an abbreviation for Ryanodone. A plus sign indicated that a particular agent is stimulatory; a minus sign indicates that a particular agent is inhibitory.

### DETAILED DESCRIPTION OF THE INVENTION

The embodiments of the invention are described with reference to the treatment of tumors in humans; however it is to be understood that the methods and compositions of the invention may be used to treat tumors in other mammals.

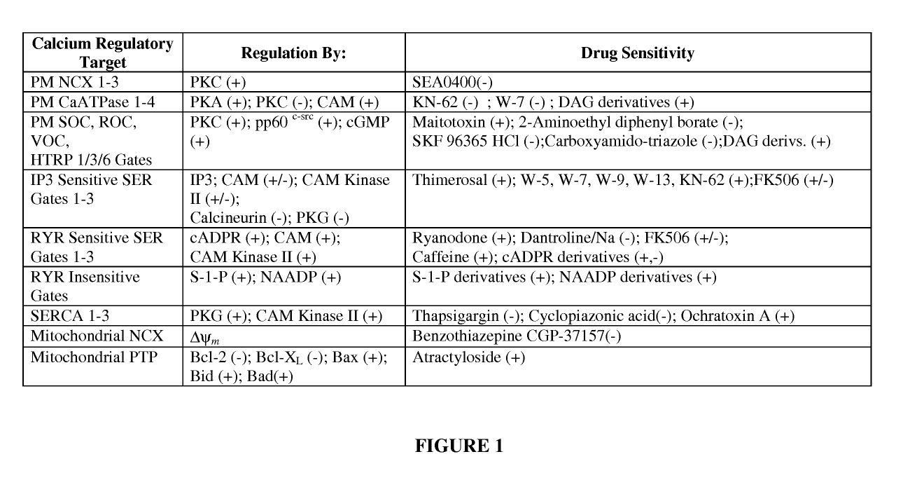

The terms “agonist” and “antagonist” as used herein are not limited to drugs acting directly on the designated targets but also encompasses drugs designed to stimulate or inhibit various elements of regulatory pathways that normally control the physiological state of plasma membrane and intracellular Ca++ gates and pumps. A summary of known protein isoforms that control Ca++ distribution within cells is shown in FIG. 1, along with exemplary drugs or regulatory molecules known to influence the activity of these proteins. These agents, or derivatives of them, are expressly contemplated as non-limiting examples of therapeutic agents for the therapeutic methods discussed in the following sections.

### A. Creation of Excessive Ca++ Filling of SER

In the case that access to occluded apoptotic effectors (e.g. DAPK) is constrained to a specific spatial pathway involving specific SER Ca sequestration sites, pharmacological manipulation is applied to promote over-filling of said depots with subsequent localized Ca leakage. Thus, in one series of embodiments, a patient with a tumor is treated with combinations of pharmacological agents that stimulate only a slight increase in the Ca burden of SER. If SER depots are nearly completely filled in tumor cells but not normal cells, using combinations of such drugs in mutually potentiating, submaximal concentrations results in a quantity of leaked Ca++ sufficient to trigger apoptosis only in the tumor cells. Suitable mechanistic examples of therapeutic drugs for these embodiments include, but are not limited to, those agents listed in FIG. 1. The drugs listed in FIG. 1 are described as follows, wherein each reference is specifically incorporated herein by reference in its entirety:

SEA0400 is described in Matsuda, T. et al (2001) J. Pharmacol. Exp. Ther. 298:249-256.
KN-62 is described in Tokumitsu et al. (1990) J. BioI. Chern. 256:4315-4320.
W-5, W-7,W-9, and W-13 are described in Hidaka et al. (1981) Proc. NatI. Acad. Sci. U.S.A. 78:4354-4357; DAG derivatives are described in Garcia-Bermejo et al. (2002) J. BioI. Chern. 277:645-655.
Maitotoxin is described in Gusovsky, F. and Daly, J. W. (1990) Biochern. Pharm 39:1633-1639.
2-Aminoethyl diphneyl borate is described in Ma, et al (2000) Science 287:1647-1651.
SKF96365 HCI and carboxyamido-triazole are described in Kohn et al. (1994) J. BioI. Chern. 269:21505-21511.
Thimerosal is described in Bird et al (1993) J. BioI. Chern. 268:17917-17923.
FK506 is described in MackriII, J. J. (1999) Biochem. J. 337:345-361.
Ryanodone is described in Sutko et al (1997) Pharmacol. Reviews 49:53-98.
DantrolinelNa is described in Zhao et al (2001) J. BioI. Chern. 276:13810-13816.
Caffeine is described in Lee, H. C. (1993) J. BioI. Chern. 268:293-299.
cADPR derivatives are described in Sethi et al. (1997) J. BioI. Chem. 272:16358-16363.
cADPR derivatives and NAADP derivatives are described in Walseth, T. F. and Lee, H. C. (2002) Lee, H. C. (ed), pp.121-142, Dordrecht: Kluwer.
S-I-P derivatives are described in Brinkmann et al (2002) J. Bioi. Chem. 277:21453-21457.
Thapsigargan is described in Davidson, G. A. and Varhol, R. J. (1995) J. Bioi. Chem. 270:11731-11734.
Cyclopiazonic acid is described in Plenge-Tellechea et al (1997) J. Bioi. Chem. 272:2794-2800.
Ochratoxin A is described in Gekle et al (2000) J. Pharm Exptl. Ther. 293: 837-844 and in Dirheimer, G. and Creppy, E. E. (1991) IARC Sci. Publ. 115:171-186.
Benzothiazepine CGP-37157 is described in Baron, K. T and Thayer, S. A. (1997) Eur. J. Pharmacol. 340:295-30.
Atractyloside is described in Zamzami et al (1996) J. Exp. Med 183:1533-1544.

In one embodiment, a patient with a tumor is treated with one or more antagonists of NCX and/or PMCA used individually or in synergistic combinations. A suitable NCX antagonist includes, but is not limited to, SEA0400. Suitable PMCA antagonists include, but are not limited to, KN-62 and W-7.

In one embodiment, a patient with a tumor is treated with one or more stimulators of SERCA in combination with one or more antagonists of SER Ca++ gates (RyR, cADPR-R, SCaMPER, IP3-R, NAADP-R) or any other SER Ca-releasing receptors. A suitable, non-limiting example of a SERCA stimulator is Ochratoxin A. Suitable antagonists of SER Ca++ gates include, but are not limited to, FK-506, Dantrolene/Na, and 7-Deaza-8-bromo-cyclic ADP-ribose.

In one embodiment, a patient with a tumor is treated with one or more stimulators of SERCA in combination with one or more antagonists of mitochondrial Ca++ uptake mechanisms. A suitable, non-limiting example of a SERCA stimulator is Ochratoxin A. A suitable non-limiting example of an antagonist of mitochondrial Ca++ uptake is Benzothiazepine CGP-37157.

In one embodiment, a patient with a tumor is treated with one or more inhibitors of plasma membrane PM Ca++ efflux pumps in any combination as follows, suitable non-limiting examples of which are cited in Table 2 and attendant references:

a) one or more antagonists of NCX and/or PMCA used individually or in synergistic combinations; and/or

b) one or more stimulators of SERCA in combination with antagonists of SER Ca++ gates (RyR, cADPR-R, SCaMPER, IP3-R, NAADP-R, or any other SER Ca-releasing receptors).

In one embodiment, a patient with a tumor is treated with one or more inhibitors of NCX, PMCA, SER Ca++ gates, SERCA agonists, and mitochondrial Ca++ uptake inhibitors in combination with one or more stimulators of NO production, a suitable, non-limiting example of the latter being exemplified by S-nitrosothiol (Al-Sa'doni, H. and Ferro, A. (2000) Clin. Sci. 98:507-520).

In one embodiment, a patient with a tumor is treated with one or more inhibitors of NCX, PMCA, SER Ca++ gates, SERCA agonists, and mitochondrial Ca++ uptake inhibitors in combination with one or more stimulators of cGMP levels, suitable, non-limiting examples of the latter being exemplified by direct (Uroguanylin or small molecular weight derivatives thereof; Pitari, G. M. et al. (2001) Proc. Natl. Acad. Sci. U.S.A. 98:7846-7851; Pitari, G. M. et al. (2003) Proc. Natl. Acad. Sci. U.S.A. 100: 2695-2699) or indirect (NO donors such as S-Nitrosothiols; Al-Sa'doni, H. and Ferro, A. (2000) Clin. Sci. 98:507-520) stimulators of GC isoforms or inhibitors of cGMP PDE (Sulindac™ sulfone or derivatives; Soh et al., (2001) J. Biol. Chem. 276:16406-16410).

In one embodiment, a patient with a tumor is treated with one or more PKC agonists (suitable, non-limiting examples of which are represented by therapeutically active derivatives of DAG as cited in Table 2) in combination with any of the therapies in the foregoing embodiments that stimulate only a slight increase in the Ca burden of SER. In this embodiment, the normal Ca storage phases of the cell cycle are prolonged or exaggerated as outlined in the background section above.

In one embodiment, a patient with a tumor is treated with one or more CAM antagonists in combination with one or more PKC agonists. This embodiment prolongs or exaggerates the normal Ca storage phases of the cell cycle while simultaneously inhibiting Ca release phases of the cell cycle.

In one embodiment, a patient with a tumor is treated with one or more agents that activate or increase the expression of mitochondrial PTP, a suitable non-limiting example of which is exemplified by Atractyloside or, although not yet developed, a small molecular weight drug that mimics the action of the pro-apoptotic Bax family of proteins, in combination with any of the therapies in the foregoing embodiments that stimulate only a slight increase in the Ca burden of SER.

### B. Selective Stimulation of Increases in Cytosolic Ca++

The disclosure further teaches, the effective concentration of cytosolic Ca++ (rather than the spatial distribution of Ca sequestration sites) is manipulated in order to trigger apoptosis in tumor cells. In cases where the SER of tumor cells carries a greater Ca burden, then only slight stimulation of Ca release at low drug concentrations releases quantitatively greater levels of Ca++ in the immediate vicinity of DAPK/NOS than what would be expected in normal cells Likewise, pharmacologically shifting the ratio of Ca++ influx to efflux by only a small extent produces apoptosis selectively in tumor cells. Suitable therapeutic agents for these embodiments include, but are not limited to, agents listed in FIG. 1 and attendant references.

In one embodiment, a patient with a tumor is treated with one or more antagonists of NCX and/or PMCA used individually or in synergistic combinations.

In one embodiment, a patient with a tumor is treated with one or more stimulators of PM Ca++ gates in combination with one or more antagonists of NCX and/or PMCA.

In one embodiment, a patient with a tumor is treated with one or more agonists of SER Ca++ release in combination with one or more antagonists of SERCA.

In one embodiment, a patient with a tumor is treated with one or more agonists of SER Ca++ release in combination with one or more inhibitors of PMCA.

In one embodiment, a patient with a tumor is treated with one or more agonists of SER Ca++ release in combination with one or more inhibitors of NCX.

In one embodiment, a patient with a tumor is treated with one or more agonists of SER Ca++ release in combination with one or more inhibitors of mitochondrial Ca++ uptake.

In one embodiment, a patient with a tumor is treated with one or more agonists of SER Ca++ release in combination with any combination of:

a) one or more antagonists of NCX and/or PMCA used individually or in synergistic combinations; and/or

b) one or more stimulators of PM Ca++ gates in combination with one or more antagonists of NCX and/or PMCA; and/or

c) one or more agonists of SER Ca++ release in combination with one or more antagonists of SERCA; and/or

d) one or more agonists of SER Ca++ release in combination with one or more inhibitors of PMCA.

In one embodiment, a patient with a tumor is treated with one or more agonists of PKC in combination with one or more antagonists of SERCA.

### C. Stimulation of Mitochondrial Ca++ Loading

The disclosure teaches apoptosis is triggered in tumor cells by treatment with drugs that stimulate mitochondrial Ca++ loading. It is expressly contemplated that mitochondrial Ca++ loading can occur over a different concentration range than those required to trigger apoptosis through non-mitochondrial pathways. Suitable, non-limiting mechanistic examples of drug categories useable for the following embodiments are sited in FIG. 1.

In one embodiment, a patient with a tumor is treated with one or more agonists of SER Ca++ release in combination with one or more agonists of PM Ca++ gates.

In one embodiment, a patient with a tumor is treated with one or more agonists of SER Ca++ release in combination with one or more antagonists of PM Ca++ efflux pumps.

In one embodiment, a patient with a tumor is treated with one or more agonists of PM Ca++ gates in combination with one or more antagonists of PM Ca++ efflux pumps.

### D. Overcoming Blocks in Apoptotic Pathways

The disclosure teaches any of the treatments outlined in Sections A. and B. above is performed in combination with DNA methylation antagonists. DNA methylation antagonists promote re-expression of DAPK in deficient tumors, (Katzenellenbogen, R.A. et al. (1999). Blood 93:4327-4353) Ultimately, the blocks in apoptotic pathways are overcome.

### E. Potentiation of Apoptosis Induced by DNA Damaging or Antimitotic Drugs

The disclosure teaches any of the treatments outlined in Sections A and B above is performed in combination with classical DNA damaging drugs or antimitotic drugs to potentiate cell killing in tumors and/or to shorten duration of treatment regimens.

### F. Exemplary Clinical Embodiment of Treatment Methodology

In each of the treatment methods provided above, there is a therapeutic window for selectively initiating an Apoptotic cascade in tumor cells without simultaneously inducing undesirable side effects in normal Ca-dependent physiological processes of normal cells. This treatment window can easily be determined by the routine experimentation of one skilled in the art. While inhibitors of plasma membrane efflux pumps may provide some clinically efficacy, employing submaximal combinations of drugs that interact synergistically to increase cellular Ca loading provides an unexpected means to reduce undesirable side effects and to increase therapeutic indices.

The duration of treatment required to initiate an Apoptotic response in patients is relatively brief, on the order of 8 to 16 hours. Individual drugs or drug combinations are administered by standard means according to the absorptive and pharmacokinetic requirements of efficacious drug candidates. The therapeutic agents are administered orally or intravenously in amounts calculated to achieve measured blood concentrations approximating those determined to be effective from tissue culture studies. Each drug would be used at the lowest dosage shown to produce mutual potentiation of apoptosis.

Blood levels of given therapeutic agents are monitored by suitable assay methods specifically developed for this purpose in order to maximize therapeutic ratios. Depending on the severity of any side effects, this treatment regimen is repeated at regular intervals as often as necessary to maximize tumor regression. In one embodiment, drug responsiveness and treatment efficacy are monitored during the course of drug administration by assay of blood levels apoptotic markers, namely any of several caspases released by cells undergoing Apoptosis specifically developed for this purpose. In this way, patients are spared unnecessarily prolonged drug exposure and the clinician is furnished with immediate evidence of treatment efficacy.

### G. High Throughput Screening for Additional Drug Candidates

High throughput drug screens can be conducted in tissue cultures of suitably matched normal and transformed human cell lines. Cells growing logarithmically may be exposed to a broad concentration range of individual drugs and drug pairs predicted to act synergistically for a period of time that represents one doubling time. The use of logarithmically growing cells presumes such cells will express isoforms of various Ca++ handling protein targets specific to the post-restriction point phases of the cell cycle. In intact populations of cells, drug responses may be measured by flow cytometry with respect to cell number, cell cycle distribution, and apoptotic fraction. Direct measurement screens of the effects of various drug combinations on specific drug targets involved in Ca++ distribution and fluxes can also be affected by those practiced in the art using confocal microscopy, Ca⁴⁵ efflux, flow cytometry, and inside-out patch clamp techniques.

### Prophetic Examples

### Example 1

A patient is administered orally a combination of 2 or 3 stable small molecule drugs (for example, activators of Protein Kinase C and inhibitors of CAM-dependent Protein Kinase II and/or Calcineurin) at synergistic and submaximal concentrations. The dosage of each drug is calculated to provide clinically effective blood levels for a period of 3 to 5 hours based on animal and Phase I trials. This short duration of treatment is based upon the minimum time required to force tumor cells into irreversible commitment to apoptosis. Resorption of a patient's tumor can be followed at appropriate intervals thereafter using ultra-sensitive techniques such as PET or SPECT molecular imaging. This regimen can be repeated daily if required based upon the severity, if any, of side-effects and by the rate of tumor shrinkage. Given the thresholds of sensitivity to calcium-induced apoptosis between normal and cancerous cells, such side-effects are likely to be fairly innocuous.

### Example 2

A patient is administered effective drug combinations by IV in order to achieve rapid and therapeutically effective blood levels that can be more precisely controlled in dosage and time in order to further reduce possible side effects. Again, drug exposure times need not be greater than 5 hours and may be as short as 3 hours based on in vitro experimental results. Such a treatment regimen can be repeated as often as necessary provided side-effects remain low or absent to ensure absolute eradication of all malignant cells any where in the body. Because the mechanisms which trigger apoptosis are also obligatory for cell cycling and because treatment regimes are so short, it is highly unlikely that mutations in tumor cells can be selected for since such mutations will also arrest cell cycle traverse. Because drug exposure times can be kept so short with even a one-time treatment being therapeutically effective, and because cells cannot develop mutational immunity to such drugs, the possibility of secondary forms of drug resistance developing, such as induction of drug efflux pumps, is minimized.

1. A method for treating a tumor in a patient comprising administering to said patient effective amounts of two or more synergistically interacting drugs that stimulate an increase in the intracellular Ca++ burden, wherein the increase is located in the cytosol, the smooth endoplasmic reticulum (SER) or the mitochondrial compartments or combinations therein.

2. The method of claim 1 wherein said drugs are antagonists of the Na/Ca antiporter (NCX).

3. The method of claim 1 wherein said drugs are antagonists of the Plasma Membrane Ca-ATPase (PMCA).

4. The method of claim 1 wherein at least one of said drugs is an antagonist of the Na/Ca antiporter (NCX) and wherein at least one of said drugs is an antagonist of the Plasma Membrane Ca-ATPase (PMCA).

5. The method of claim 1 wherein at least one of said drugs stimulates Sarco-Endoplasmic-Reticulum Ca-ATPase (SERCA) and wherein at least one of said drugs is an antagonist of SER Ca++ gates.

6. (canceled)

7. (canceled)

8. . The method of claim 1 wherein at least one of said drugs is selected from the group consisting of inhibitors of SER Ca++ gates and SERCA agonists; and one of the said drugs is a stimulator of Nitric Oxide (NO) production.

9. The method of claim 1 wherein at least one of said drugs is selected from the group consisting of inhibitors of SER Ca++ gates and SERCA agonists; and one of said drugs is selected from the group consisting of drugs that effectively elevate cGMP levels and stable mimetic analogs of cGMP.

10. The method of claim 1 wherein at least one of said drugs is a Calmodulin (CAM) antagonist and wherein at least one of said drugs is a Protein Kinase C (PKC) agonist.

11. The method of claim 10 wherein the Calmodulin antagonist is selected from the group consisting of antagonists of Calcineurin and antagonists of CAM-dependent Protein Kinase II.

12. The method of claim 10 wherein the Calmodulin antagonist is selected from the group consisting of the cyclosporine A class of drugs, cell permeable Calcineurin autoinhibitory domain poly-arginine-based polypeptide, and direct small molecule inhibitors of PP2B Protein Phosphatase.

13. The method of claim 10 wherein said Protein Kinase C agonist is ceramide C6 or derivatives thereof.

14. A method of treating a tumor in a patient comprising administering to said patient effective amounts of two or more drugs that stimulate mitochondrial Ca++ loading.

15. The method of claim 14 wherein at least one of said drugs is an agonist of SER Ca++ release and wherein at least one of said drugs is an agonist of PM Ca++ gates.

16. The method of claim 14 wherein at least one of said drugs is an agonist of SER Ca++ release and wherein at least one of said drugs is an antagonist of PM Ca++ efflux pumps.

17. The method of claim 1 further comprising administering to said patient an effective amount of a DNA damaging agent.

18. The method of claim 14 further comprising administering to said patient an effective amount of a DNA damaging agent.

19. The method of claim 1 further comprising administering to said patient an effective amount of an anti-mitotic drug.

20. The method of claim 14 further comprising administering to said patient an effective amount of an anti-mitotic drug.

21. The method of claim 9 wherein one of the drugs selected is a guanylate cyclase agonist.

22. The method of claim 9 wherein one of the drugs selected is a cyclic GMP Phosphodiesterase antagonist.

23. The method of claim 11 wherein the Calmodulin antagonist is an inhibitor of Protein Phosphatase 2B.

24. The method of claim 12 wherein the Calmodulin antagonist is an inhibitor of CAM-dependent Protein Kinase II.

25. The method of claims 23 wherein the antagonist is KN-62.
